(19) United States
(12) Patent Application Publication
(10) Pub. No.: US 2023/0230398 A1
(43) Pub. Date: Jul. 20, 2023

(54) IMAGE PROCESSING DEVICE, IMAGE PROCESSING METHOD, IMAGE PROCESSING PROGRAM, AND DIAGNOSIS SUPPORT SYSTEM

(71) Applicant: SONY GROUP CORPORATION, TOKYO (JP)

(72) Inventors: YOHEI KOBAYASHI, TOKYO (JP); TAKESHI KUNIHIRO, Tokyo (JP); JUNICHIRO ENOKI, Tokyo (JP); KENJI YAMANE, Tokyo (JP); YUTAKA HASEGAWA, Tokyo (JP)

(21) Appl. No.: 18/002,423

(22) PCT Filed: Jun. 2, 2021
(86) PCT No.: PCT/JP2021/020921

§ 371 Date: Dec. 19, 2022

(30) Foreign Application Priority Data
Jun. 26, 2020 (JP) 2020-110156

(51) Int. Cl. G06V 20/69; G06V 10/44; G06V 10/22; G06T 7/73; G06T 7/00; G06V 10/77; G16H 30/40; G06T 7/11

(52) U.S. Cl. CPC G06V 20/695; G06V 10/44; G06V 10/22; G06T 7/73; G06T 7/0012; G06V 10/7715; G06V 20/698; G16H 30/40; G06T 7/11; G06T 2207/10056; G06V 2201/03; G06T 2207/30096; G06T 2207/30024

(57) ABSTRACT

An image processing device 100 includes, in a case where designation of a plurality of partial regions corresponding to a cell morphology is received, the plurality of partial regions being extracted from a pathological image, a generation unit 154 that generates auxiliary information indicating information about a feature amount effective when a plurality of partial regions is classified or extracted with respect to a plurality of feature amounts calculated from the image; and in a case where setting information about an adjustment item according to the auxiliary information is received, an image processing unit 155 that performs an image process on the image using the setting information.

### FIELD

The present invention relates to an image processing device, an image processing method, an image processing program, and a diagnosis support system.

### BACKGROUND

There is a system that photographs an observation target placed on a glass slide with a microscope, generates a digitized pathological image, and performs various types of image analyses on the pathological image. For example, the observation target is a tissue or a cell collected from a patient, and corresponds to a piece of meat of an organ, saliva, blood, or the like.

As a conventional technique related to image analysis, a technique of inputting a pathological image to a morphology detector, detecting a morphology or a state of a cell nucleus, a cell membrane, or the like included in the pathological image, and calculating a feature amount obtained by quantifying a feature of the morphology is known. A skilled person such as a pathologist or a researcher sets adjustment items of an identifier for classifying or extracting a morphology or a state having a specific feature based on the calculation result of the feature amount and the specialized knowledge.

### CITATION LIST

### Patent Literature

Patent Literature 1: JP 2018-502279 W

### SUMMARY

### Technical Problem

It is difficult for a user with poor specialized knowledge to associate a feature amount obtained by quantifying a feature of a morphology and a state calculated by the prior art with a feature based on the specialized knowledge of the user, and there is room for improvement.

Therefore, the present disclosure proposes an image processing device, an image processing method, an image processing program, and a diagnosis support system capable of appropriately displaying a feature amount obtained by quantifying an appearance feature of a morphology and easily setting an adjustment item of an identifier.

### Solution to Problem

To solve the problems described above, an image processing device according to an embodiment of the present disclosure includes: in a case where designation of a plurality of partial regions corresponding to a cell morphology is received, the plurality of partial regions being extracted from a pathological image, a generation unit that generates auxiliary information indicating information about a feature amount effective when a plurality of partial regions is classified or extracted with respect to a plurality of feature amounts calculated from the image; and in a case where setting information about an adjustment item according to the auxiliary information is received, an image processing unit that performs an image process on the image using the setting information.

### BRIEF DESCRIPTION OF DRAWINGS

FIG. 1 is a diagram illustrating a diagnosis support system according to the present embodiment.

FIG. 2 is a diagram for explaining an imaging process according to the present embodiment.

FIG. 3 is a diagram for explaining an imaging process according to the present embodiment.

FIG. 4 is a diagram for explaining a process of generating a partial image (tile image).

FIG. 5 is a diagram for explaining a pathological image according to the present embodiment.

FIG. 6 is a diagram for explaining a pathological image according to the present embodiment.

FIG. 7 is a diagram illustrating an example of a browsing mode by a viewer of a pathological image.

FIG. 8 is a diagram illustrating an example of a browsing history storage unit included in a server.

FIG. 9A is a diagram illustrating a diagnostic information storage unit included in a medical information system.

FIG. 9B is a diagram illustrating a diagnostic information storage unit included in the medical information system.

FIG. 9C is a diagram illustrating a diagnostic information storage unit included in the medical information system.

FIG. 10 is a diagram illustrating an example of an image processing device according to the present embodiment.

FIG. 11 is a diagram illustrating an example of a data structure of a pathological image DB.

FIG. 12 is a diagram illustrating an example of a data structure of a feature amount table.

FIG. 13 is a diagram illustrating an example of a partial region extracted from a pathological image.

FIG. 14 is a diagram for explaining a process by a display control unit.

FIG. 15 is a diagram illustrating an example of first auxiliary information.

FIG. 16 is a diagram illustrating an example of second auxiliary information.

FIG. 17 is a diagram illustrating another display example of the second auxiliary information.

FIG. 18 is a diagram illustrating an example of third auxiliary information.

FIG. 19 is a diagram illustrating an example of fourth auxiliary information.

FIG. 20 is a diagram for explaining an example of a classification process performed by an image processing unit.

FIG. 21 is a diagram for explaining an example of a classification process performed by an image processing unit.

FIG. 22 is a diagram for explaining an example of a classification process performed by an image processing unit.

FIG. 23 is a flowchart illustrating a processing procedure of the image processing device according to the present embodiment.

FIG. 24 is a diagram for explaining another process by the image processing device.

FIG. 25 is a diagram for explaining another process by the image processing device.

FIG. 26 is a hardware configuration diagram illustrating an example of a computer that implements functions of an image processing device.

### DESCRIPTION OF EMBODIMENTS

Hereinafter, the embodiments of the present disclosure will be described in detail with reference to the drawings. In the following embodiments, the same parts are denoted by the same reference signs, and a duplicate description will be omitted.

Further, the present disclosure will be described in the order of the following items.

<Present Embodiment>

1. Configuration of system according to present embodiment

2. Various kinds of information

2-1. Pathological image

2-2. Browsing history information

2-3. Diagnostic information

3. Image processing device according to present embodiment

4. Processing procedure

5. Another process

6. Effects of image processing device according to present embodiment

7. Hardware configuration

8. Conclusion

### Present Embodiment

### 1. Configuration of System According to Present Embodiment

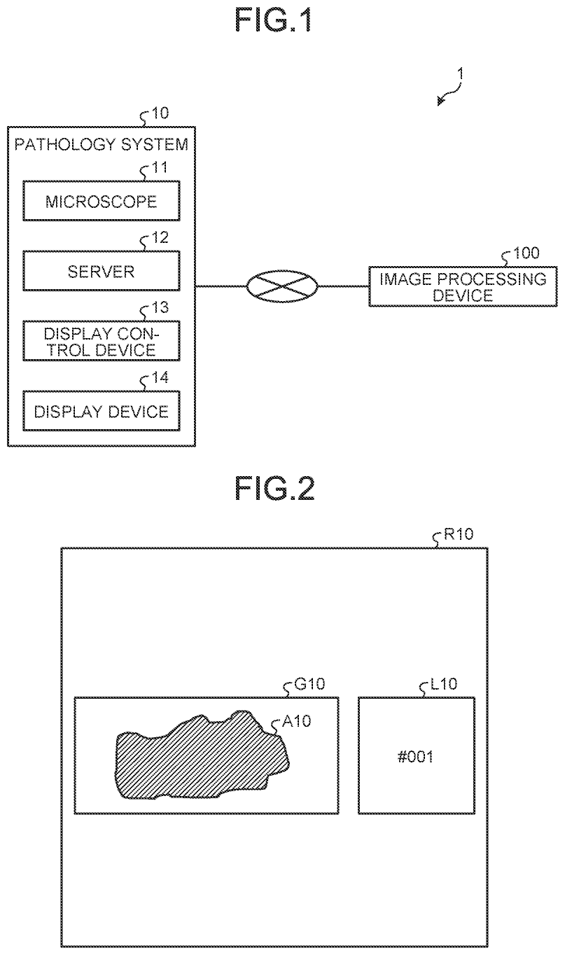

First, a diagnosis support system 1 according to the present embodiment will be described with reference to FIG. 1. FIG. 1 is a diagram illustrating a diagnosis support system 1 according to the present embodiment. As illustrated in FIG. 1, the diagnosis support system 1 includes a pathology system 10 and an image processing device 100.

The pathology system 10 is a system mainly used by a pathologist, and is applied to, for example, a laboratory or a hospital. As illustrated in FIG. 1, the pathology system 10 includes a microscope 11, a server 12, a display control device 13, and a display device 14.

The microscope 11 is an imaging device that has a function of an optical microscope, captures an image of an observation target placed on a glass slide, and acquires a pathological image that is a digital image. Note that the observation target is, for example, a tissue or a cell collected from a patient, and is a piece of meat of an organ, saliva, blood, or the like.

The server 12 is a device that stores and holds a pathological image captured by the microscope 11 in a storage unit (not illustrated). In a case of receiving the browsing request from the display control device 13, the server 12 searches for a pathological image from a storage unit (not illustrated) and transmits the searched pathological image to the display control device 13. In addition, in a case of receiving the acquisition request for the pathological image from the image processing device 100, the server 12 searches for the pathological image from the storage unit and transmits the searched pathological image to the image processing device 100.

The display control device 13 transmits a browsing request for the pathological image received from the user to the server 12. Then, the display control device 13 causes the display device 14 to display the pathological image received from the server 12.

The display device 14 has a screen using, for example, liquid crystal, electro-luminescence (EL), cathode ray tube (CRT), or the like. The display device 14 may be compatible with 4K or 8K, or may be formed by a plurality of display devices. The display device 14 displays the pathological image controlled by the display control device 13. Note that, although details will be described later, the server 12 stores the browsing history information about the region of the pathological image observed by the pathologist via the display device 14.

The image processing device 100 is a device that transmits an acquisition request for a pathological image to the server 12 and performs the image process on the pathological image received from the server 12.

### 2. Various Kinds of Information

[2-1. Pathological Image]

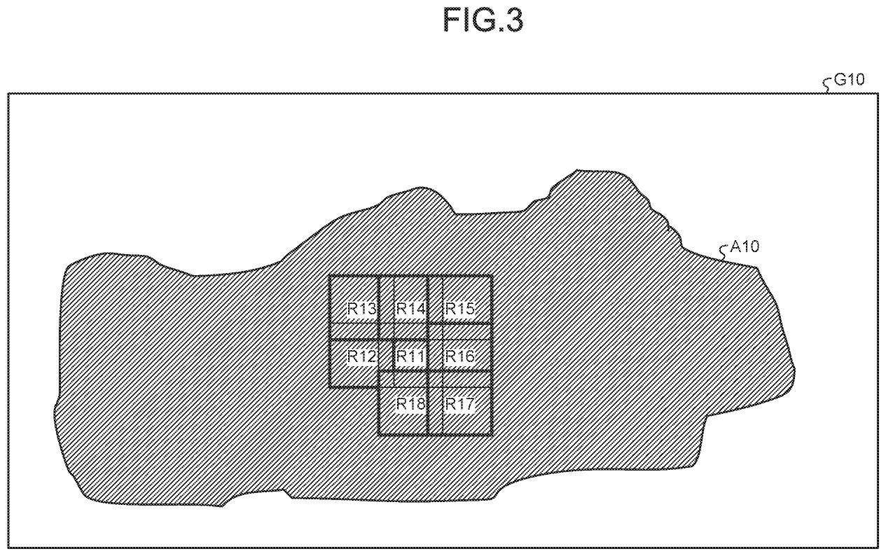

As described above, the pathological image is generated by imaging the observation target with the microscope 11. First, an imaging process by the microscope 11 will be described with reference to FIGS. 2 and 3. FIGS. 2 and 3 are diagrams for explaining the imaging process according to the first embodiment. The microscope 11 described below includes a low-resolution imaging unit for performing imaging at low resolution and a high-resolution imaging unit for performing imaging at high resolution.

In FIG. 2, a glass slide G10 on which an observation target A10 is placed is included in an imaging region R10 that is a region that can be imaged by the microscope 11. The glass slide G10 is placed on a stage (not illustrated), for example. The microscope 11 images the imaging region R10 by the low-resolution imaging unit to generate an entire image that is a pathological image in which the observation target A10 is entirely imaged. In label information L10 illustrated in FIG. 2, identification information (for example, a character string or a QR code (registered trademark)) for identifying the observation target A10 is described. By associating the identification information described in the label information L10 with the patient, it is possible to identify the patient corresponding to the entire image. In the example of FIG. 2, “#001” is described as the identification information. In the label information L10, for example, a simple description of the observation target A10 may be described.

Subsequently, after generating the entire image, the microscope 11 identifies the region where the observation target A10 exists from the entire image, and sequentially images, by the high-resolution imaging unit, each divided region obtained by dividing the region where the observation target A10 exists for each predetermined size. For example, as illustrated in FIG. 3, the microscope 11 first images a region R11 to generate a high-resolution image I11 that is an image illustrating a partial region of the observation target A10. Subsequently, the microscope 11 moves the stage to image a region R12 by the high-resolution imaging unit to generate a high-resolution image I12 corresponding to the region R12. Similarly, the microscope 11 generates high-resolution images I13, I14, . . . , corresponding to regions R13, R14, . . . , respectively. Although regions up to the region R18 are illustrated in FIG. 3, the microscope 11 sequentially moves the stage to image all the divided regions corresponding to the observation target A10 by the high-resolution imaging unit to generate the high-resolution images corresponding to the respective divided regions.

When the stage is moved, the glass slide G10 may move on the stage. When the glass slide G10 moves, an unimaged region of the observation target A10 may occur. As illustrated in FIG. 3, the microscope 11 captures an image by the high-resolution imaging unit such that the adjacent divided regions partially overlap each other, and thus, it is possible to prevent the occurrence of an unimaged region even when the glass slide G10 moves.

Note that the low-resolution imaging unit and the high-resolution imaging unit described above may be different optical systems or may be the same optical system. In a case of the same optical system, the microscope 11 changes the resolution according to the imaging target. Furthermore, in the above description, an example is described in which the imaging region is changed by moving the stage, but the imaging region may be changed by the microscope 11 moving an optical system (high-resolution imaging unit or the like). The imaging element provided in the high-resolution imaging unit may be a two-dimensional imaging element (area sensor) or a one-dimensional imaging element (line sensor). The light from the observation target may be condensed and imaged using the objective lens, or may be dispersed and imaged for each wavelength using the spectroscopic optical system. In addition, FIG. 3 illustrates an example in which the microscope 11 captures an image from the central portion of the observation target A10. However, the microscope 11 may image the observation target A10 in an order different from the imaging order illustrated in FIG. 3. For example, the microscope 11 may capture an image from the outer peripheral portion of the observation target A10. Furthermore, in the above description, an example is described in which only the region where the observation target A10 exists is imaged by the high-resolution imaging unit. However, since there is a case where the region where the observation target A10 exists cannot be accurately extracted, the microscope 11 may divide the entire region of the imaging region R10 or the glass slide G10 illustrated in FIG. 2 and image the divided regions by the high-resolution imaging unit. Note that any method may be used as a method of capturing a high-resolution image. The divided region may be imaged to acquire a high-resolution image while repeating stop and movement of the stage, or the divided region may be imaged to acquire a high-resolution image on the strip while moving the stage at a predetermined speed.

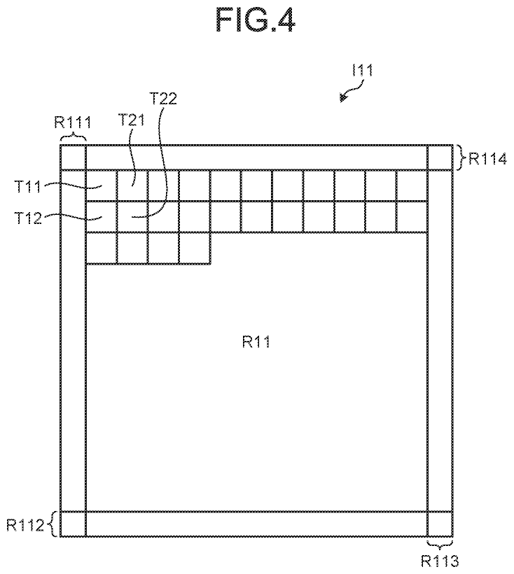

Subsequently, each high-resolution image generated by the microscope 11 is divided into predetermined sizes. As a result, a partial image (hereinafter, it is referred to as a tile image.) is generated from the high-resolution image. This point will be described with reference to FIG. 4. FIG. 4 is a diagram for describing a process of generating a partial image (tile image). FIG. 4 illustrates the high-resolution image I11 corresponding to the region R11 illustrated in FIG. 3. Note that, in the following description, it is assumed that a partial image is generated from a high-resolution image by the server 12. However, the partial image may be generated by a device (for example, an information processing device mounted inside the microscope 11, or the like.) other than the server 12.

In the example illustrated in FIG. 4, the server 12 generates 100 tile images T11, T12, . . . , by dividing one high-resolution image I11. For example, in a case where the resolution of the high-resolution image I11 is 2560×2560 [pixel], the server 12 generates 100 tile images T11, T12, . . . , each having a resolution of 256×256 [pixel], from the high-resolution image I11. Similarly, the server 12 generates tile images by dividing other high-resolution images by the similar size.

Note that, in the example of FIG. 4, regions R111, R112, R113, and R114 are regions overlapping with adjacent high-resolution images (not illustrated in FIG. 4). The server 12 performs positioning of the overlapping regions by a technique such as template matching to perform a stitching process on the adjacent high-resolution images. In this case, the server 12 may generate the tile images by dividing the high-resolution image after the stitching process. Alternatively, the server 12 may generate the tile images of regions other than the regions R111, R112, R113, and R114 before the stitching process, and generate the tile images of the regions R111, R112, R113, and R114 after the stitching process.

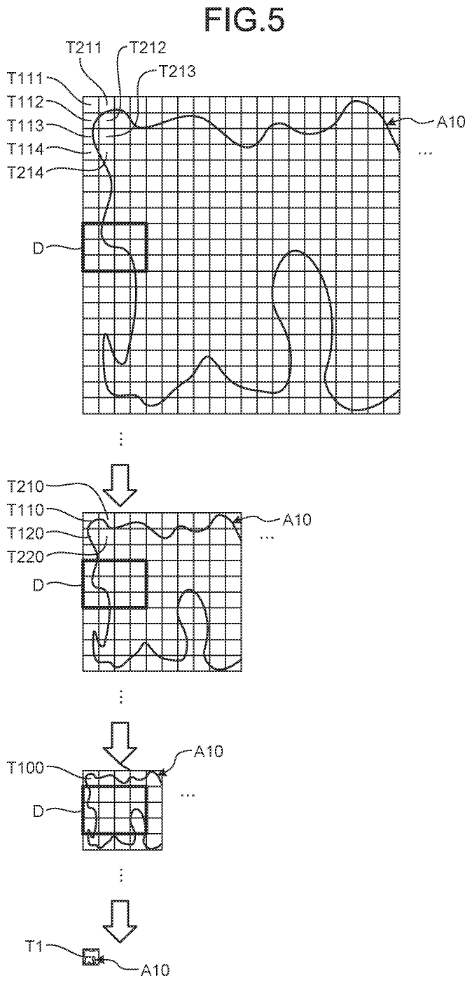

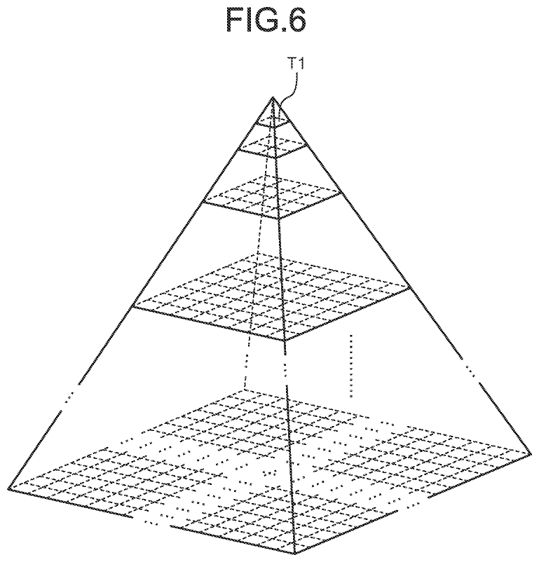

In this manner, the server 12 generates the tile image that is the minimum unit of the captured image of the observation target A10. Then, the server 12 sequentially combines the tile images of the minimum unit to generate the tile images having different hierarchies. Specifically, the server 12 generates one tile image by combining a predetermined number of adjacent tile images. This point will be described with reference to FIGS. 5 and 6. FIGS. 5 and 6 are diagrams for explaining a pathological image according to the first embodiment.

The upper part of FIG. 5 illustrates a tile image group of a minimum unit generated from each high-resolution image by the server 12. In the example in the upper part of FIG. 5, the server 12 generates one tile image T110 by combining four tile images T111, T112, T211, and T212 adjacent to each other among the tile images. For example, in a case where the resolution of each of the tile images T111, T112, T211, and T212 is 256×256, the server 12 generates the tile image T110 having a resolution of 256×256. Similarly, the server 12 generates a tile image T120 by combining four tile images T113, T114, T213, 1214 adjacent to each other. In this manner, the server 12 generates the tile images obtained by combining a predetermined number of tile images of the minimum unit.

Furthermore, the server 12 generates a tile image obtained by further combining tile images adjacent to each other among the tile images obtained by combining the tile images of the minimum unit. In the example of FIG. 5, the server 12 generates one tile image T100 by combining four tile images T110, T120, T210, and T220 adjacent to each other. For example, in a case where the resolution of each of the tile images T110, T120, T210, and T220 is 256×256, the server 12 generates the tile image T100 having a resolution of 256×256. For example, the server 12 generates an image having a resolution of 256×256 by performing 4-pixel averaging process, a weighting filtering process (processing of reflecting close pixels more strongly than far pixels), a ½ thinning processing, or the like from a tile image having a resolution of 512×512 obtained by combining four tile images adjacent to each other.

By repeating such a combining process, the server 12 finally generates one tile image having a resolution similar to the resolution of the tile image of the minimum unit. For example, as in the above example, in a case where the resolution of the tile image of the minimum unit is 256×256, the server 12 generates one tile image T1 having a resolution of 256×256 finally by repeating the above-described combining process.

FIG. 6 schematically illustrates the tile image illustrated in FIG. 5. In the example illustrated in FIG. 6, the lowermost layer tile image group is tile images of a minimum unit generated by the server 12. Furthermore, the tile image group in the second hierarchy from the bottom is tile images after the lowermost tile image group is combined. Then, the uppermost tile image T1 indicates one tile image to be finally generated. In this way, the server 12 generates tile image groups having a hierarchy such as a pyramid structure illustrated in FIG. 6 as the pathological image.

Note that a region D illustrated in FIG. 5 is an example of a region displayed on a display screen of display device 14 or the like. For example, it is assumed that the resolution displayable by the display device corresponds to vertically three tile images and horizontally four tile images. In this case, as in a region D illustrated in FIG. 5, the level of detail of the observation target A10 displayed on the display device varies depending on the hierarchy to which the tile image to be displayed belongs. For example, in a case where the lowermost layer tile image is used, a narrow region of the observation target A10 is displayed in detail. In addition, the wider region of the observation target A10 is coarsely displayed as the upper layer tile image is used.

The server 12 stores the tile images of the respective hierarchies as illustrated in FIG. 6 in a storage unit (not illustrated). For example, the server 12 stores each tile image together with tile identification information (an example of partial image information) that can uniquely identify each tile image. In this case, in a case of receiving an acquisition request for the tile image including tile identification information from another device (for example, the display control device 13), the server 12 transmits a tile image corresponding to the tile identification information to the another device. Furthermore, for example, the server 12 may store each tile image together with hierarchy identification information for identifying each hierarchy and tile identification information uniquely identifiable in the same hierarchy. In this case, in a case of receiving the acquisition request for the tile image including the hierarchy identification information and the tile identification information from another device, the server 12 transmits the tile image corresponding to the tile identification information among the tile images belonging to the hierarchy corresponding to the hierarchy identification information to the another device.

Note that the server 12 may store the tile images of the respective hierarchies as illustrated in FIG. 6 in a storage device other than the server 12. For example, the server 12 may store tile images of each hierarchy in a cloud server or the like. Furthermore, the process of generating tile images illustrated in FIGS. 5 and 6 may be performed by a cloud server or the like.

Furthermore, the server 12 may not store the tile images of all the hierarchies. For example, the server 12 may store only the tile images of the lowermost layer, may store only the tile images of the lowermost layer and the tile images of the uppermost layer, or may store only the tile images of a predetermined hierarchy (for example, odd-numbered hierarchies, even-numbered hierarchies, and the like.). At this time, in a case where the tile images of an unstored hierarchy is requested from another device, the server 12 generates a tile image requested from another device by dynamically combining the stored tile images. In this manner, the server 12 can prevent the storage capacity from being overloaded by thinning out the tile images to be stored.

Furthermore, although the imaging conditions are not mentioned in the above example, the server 12 may store the tile images of the respective hierarchies as illustrated in FIG. 6 for each imaging condition. An example of the imaging condition includes a focal length with respect to a subject (such as the observation target A10). For example, the microscope 11 may capture images of the same subject while changing the focal length. In this case, the server 12 may store the tile images of respective hierarchies as illustrated in FIG. 6 for each focal length. Note that the reason for changing the focal length is that, since the observation target A10 is translucent depending on the observation target A10, there are a focal length suitable for imaging the surface of the observation target A10 and a focal length suitable for imaging the inside of the observation target A10. In other words, the microscope 11 can generate a pathological image obtained by imaging the surface of the observation target A10 and a pathological image obtained by imaging the inside of the observation target A10 by changing the focal length.

In addition, another example of the imaging condition includes a staining condition for the observation target A10. Specifically, in the pathological diagnosis, a specific portion (for example, a cell nucleus or the like) of the observation target A10 may be stained using a fluorescent reagent. The fluorescent reagent is, for example, a substance that is excited and emits light when irradiated with light of a specific wavelength. Then, different luminescent substances may be stained for the same observation target A10. In this case, the server 12 may store tile images of respective hierarchies as illustrated in FIG. 6 for each dyed luminescent substance.

Furthermore, the number and resolution of the tile images described above are merely examples, and can be appropriately changed depending on the system. For example, the number of tile images combined by the server 12 is not limited to four. For example, the server 12 may repeat a process of combining 3×3=9 tile images. In the above example, the resolution of the tile image is 256×256, but the resolution of the tile image may be other than 256×256.

The display control device 13 extracts a desired tile image from the tile image group having the hierarchical structure according to an input operation of the user via the display control device 13 using software of a system capable of handling the tile image group having the hierarchical structure described above to output the extracted tile image to the display device 14. Specifically, the display device 14 displays an image of an arbitrary portion selected by the user among images of aby resolution selected by the user. By such a process, the user can obtain a feeling as if the user is observing the observation target while changing the observation magnification. That is, the display control device 13 functions as a virtual microscope. The virtual observation magnification here actually corresponds to the resolution.

[2-2. Browsing History Information]

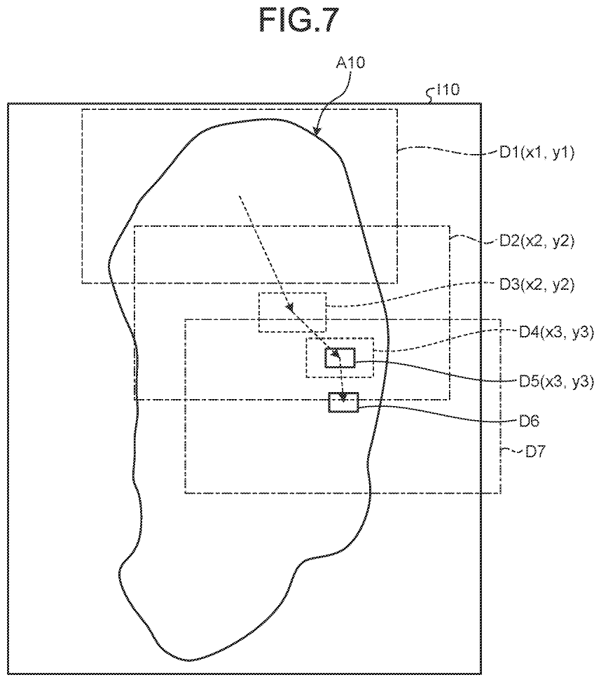

Next, the browsing history information about the pathological image stored in the server 12 will be described with reference to FIG. 7. FIG. 7 is a diagram illustrating an example of a browsing mode by a viewer of a pathological image. In the example illustrated in FIG. 7, it is assumed that a viewer such as a pathologist has browsed the regions D1, D2, D3, . . . , and D7 in this order in a pathological image I10. In this case, the display control device 13 first acquires the pathological image corresponding to the region D1 from the server 12 according to the browsing operation by the viewer. In response to a request from the display control device 13, the server 12 acquires one or more tile images forming the pathological image corresponding to the region D1 from the storage unit to transmit the acquired one or more tile images to the display control device 13. Then, the display control device 13 displays the pathological image formed from the one or more tile images acquired from the server 12 on the display device 14. For example, in a case where there is a plurality of tile images, the display control device 13 displays the plurality of tile images side by side. Similarly, each time the viewer performs the operation of changing the display region, the display control device 13 acquires the pathological image corresponding to the region to be displayed (regions D2, D3, . . . , D7, etc.) from the server 12 and displays the acquired pathological image on the display device 14.

In the example of FIG. 7, since the viewer first browses the relatively wide region D1 and there is no region to be carefully observed in the region D1, the viewer moves the browsing region to the region D2. Then, since there is a region desired to be carefully observed in the region D2, the viewer browses the region D3 by enlarging a partial region of the region D2. Then, the viewer further moves the browsing region to the region D4 which is a partial region of the region D2. Then, since there is a region desired to be observed more carefully in the region D4, the viewer browses the region D5 by enlarging a partial region of the region D4. In this manner, the viewer is also viewing the regions D6 and D7. For example, each of the pathological images corresponding to the regions D1, D2, and D7 is a display image with a 1.25-fold magnification, each of the pathological images corresponding to the regions D3 and D4 is a display image with a 20-fold magnification, and each of the pathological images corresponding to the regions D5 and D6 is a display image with a 40-fold magnification. The display control device 13 acquires and displays the tile image of the hierarchy corresponding to each magnifications in the tile image group of the hierarchical structure stored in the server 12. For example, the hierarchy of the tile images corresponding to the regions D1 and D2 is higher (that is, a hierarchy close to the tile image T1 illustrated in FIG. 6) than the hierarchy of the tile images corresponding to the region D3.

While the pathological image is browsed as described above, the display control device 13 acquires the browsing information at a predetermined sampling cycle. Specifically, the display control device 13 acquires the center coordinates and the display magnification of the browsed pathological image at each predetermined timing, and stores the acquired browsing information in the storage unit of the server 12.

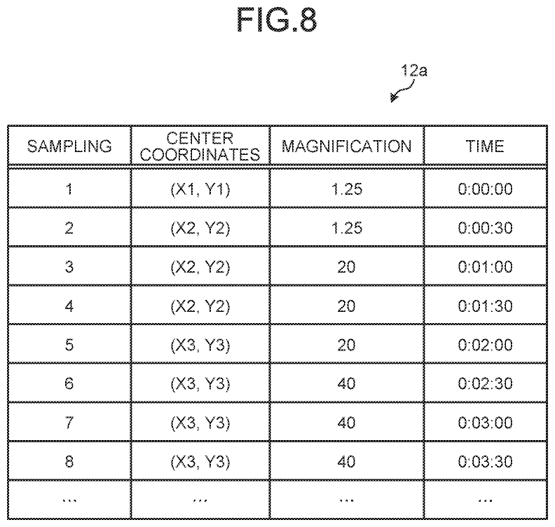

This point will be described with reference to FIG. 8. FIG. 8 is a diagram illustrating an example of a browsing history storage unit 12a included in the server 12. As illustrated in FIG. 8, the browsing history storage unit 12a stores information such as “sampling”, “center coordinates”, “magnification”, and “time”. The “sampling” indicates an order of timing of storing the browsing information. The “center coordinates” indicate positional information about the browsed pathological image. In this example, the center coordinates are coordinates indicated by the center position of the browsed pathological image, and correspond to the coordinates of the coordinate system of the lowermost layer tile image group. The “magnification” indicates a display magnification of the browsed pathological image. The “time” indicates an elapsed time from the start of browsing. The example of FIG. 8 illustrates that the sampling cycle is 30 seconds. That is, the display control device 13 stores the browsing information in the browsing history storage unit 12a every 30 seconds. However, the present invention is not limited to this example, and the sampling cycle may be, for example, 0.1 to 10 seconds, or may be out of this range.

In the example of FIG. 8, the sampling “1” indicates the browsing information about the region D1 illustrated in FIG. 7, the sampling “2” indicates the browsing information about the region D2, the samplings “3” and “4” indicate the browsing information about the region D3, the sampling “5” indicates the browsing information about the region D4, and the samplings “6”, “7”, and “8” indicate the browsing information about the region D5. That is, the example of FIG. 8 illustrates that the region D1 is browsed for about 30 seconds, the region D2 is browsed for about 30 seconds, the region D3 is browsed for about 60 seconds, the region D4 is browsed for about 30 seconds, and the region D5 is browsed for about 90 seconds. In this manner, the browsing time of each region can be extracted from the browsing history information.

Furthermore, the number of times each region has been browsed can be extracted from the browsing history information. For example, it is assumed that the number of times of display of each pixel of the displayed pathological image is increased by one each time a display region changing operation (for example, an operation of moving the display region and an operation of changing the display size) is performed. For example, in the example illustrated in FIG. 7, in a case where the region D1 is first displayed, the number of times of display of each pixel included in the region D1 is one. Next, in a case where the region D2 is displayed, the number of times of display of each pixel included in both the region D1 and the region D2 is two, and the number of times of display of each pixel included in the region D2 and not included in the region D1 is one. Since the display region can be identified by referring to the center coordinates and the magnification of the browsing history storage unit 12a, the number of times each pixel (that can also be referred to as each coordinates) of the pathological image is displayed can be extracted by analyzing the browsing history information stored in the browsing history storage unit 12a.

In a case where the operation of changing the display position is not performed by the viewer for a predetermined time (for example, 5 minutes), the display control device 13 may suspend the storage process of the browsing information. Furthermore, in the above example, an example is described in which the browsed pathological image is stored as the browsing information by the center coordinates and the magnification, but the present invention is not limited to this example, and the browsing information may be any information as long as it can identify the region of the browsed pathological image. For example, the display control device 13 may store, as the browsing information about the pathological image, tile identification information for identifying the tile image corresponding to the browsed pathological image or information indicating the position of the tile image corresponding to the browsed pathological image. Furthermore, although not illustrated in FIG. 8, information for identifying a patient, a medical record, and the like is stored in the browsing history storage unit 12a. That is, the browsing history storage unit 12a illustrated in FIG. 8 stores the browsing information, the patient, the medical record, and the like in association with each other.

[2-3. Diagnostic Information]

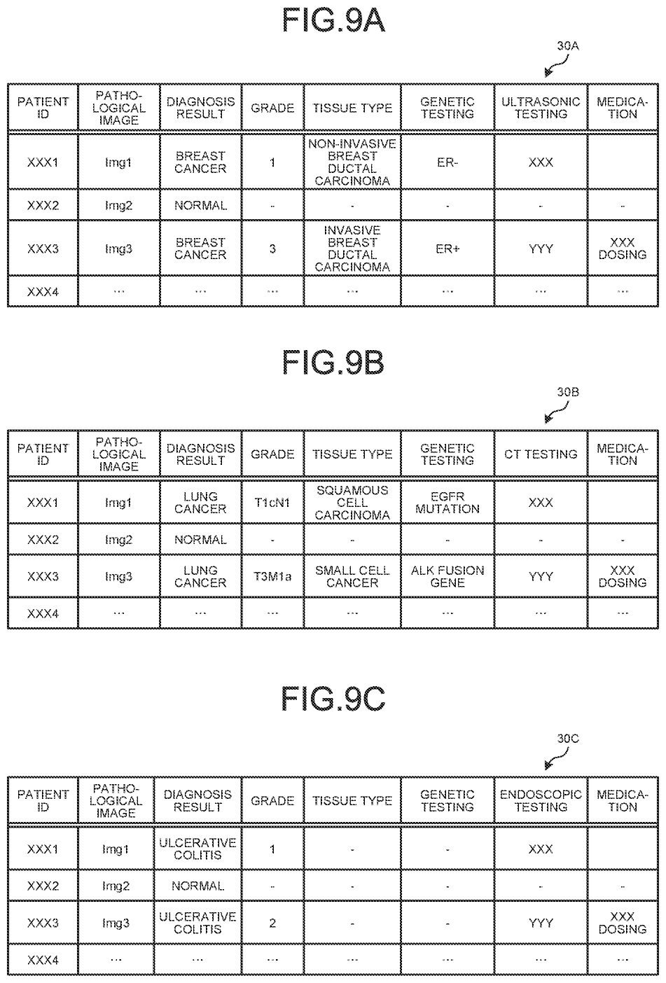

Next, diagnostic information stored in a medical information system 30 will be described with reference to FIGS. 9A to 9C. FIGS. 9A to 9C are diagrams illustrating a diagnostic information storage unit included in the medical information system 30. FIGS. 9A to 9C illustrate examples in which diagnostic information is stored in different tables for respective organs to be examined. For example, FIG. 9A illustrates an example of a table storing diagnostic information related to a breast cancer examination, FIG. 9B illustrates an example of a table storing diagnostic information related to a lung cancer examination, and FIG. 9C illustrates an example of a table storing diagnostic information related to a large intestine examination.

A diagnostic information storage unit 30A illustrated in FIG. 9A stores information such as a “patient ID”, a “pathological image”, a “diagnostic result”, a “grade”, a “tissue type”, a “genetic testing”, an “ultrasonic testing”, and a “medication”. The “patient ID” indicates identification information for identifying a patient. The “pathological image” indicates a pathological image stored by the pathologist at the time of diagnosis. In the “pathological image”, positional information (center coordinates, magnification, and the like) indicating an image region to be saved with respect to the entire image may be stored instead of the image itself. The “diagnosis result” is a diagnosis result by a pathologist, and indicates, for example, the presence or absence of a lesion site and the type of lesion site. The “grade” indicates a degree of progression of the diseased site. The “tissue type” indicates a type of diseased site. The “genetic testing” refers to a result of the genetic testing. The “ultrasonic testing” indicates a result of the ultrasonic testing. The medication indicates information about dosing to the patient.

A diagnostic information storage unit 30B illustrated in FIG. 9B stores information related to a “CT testing” performed for lung cancer examination instead of the “ultrasonic testing” stored in the diagnostic information storage unit 30A illustrated in FIG. 9A. A diagnostic information storage unit 30C illustrated in FIG. 9C stores information related to an “endoscopic testing” performed for the large intestine examination instead of the “ultrasonic testing” stored in the diagnostic information storage unit 30A illustrated in FIG. 9A.

In the examples of FIGS. 9A to 9C, in a case where the “normal” is stored in the “diagnosis result”, it indicates that the result of the pathological diagnosis is negative, and when information other than the “normal” is stored in the “diagnosis result”, it indicates that the result of the pathological diagnosis is positive. Note that, in FIGS. 9A to 9C, the case of storing the patient ID in association with respective items (pathological image, diagnostic result, grade, tissue type, genetic testing, ultrasonic testing, medication) is described. However, it is sufficient to store information related to diagnosis and testing in association with the patient ID, and not all the items are necessary.

### 3. Image Processing Device According to Present Embodiment

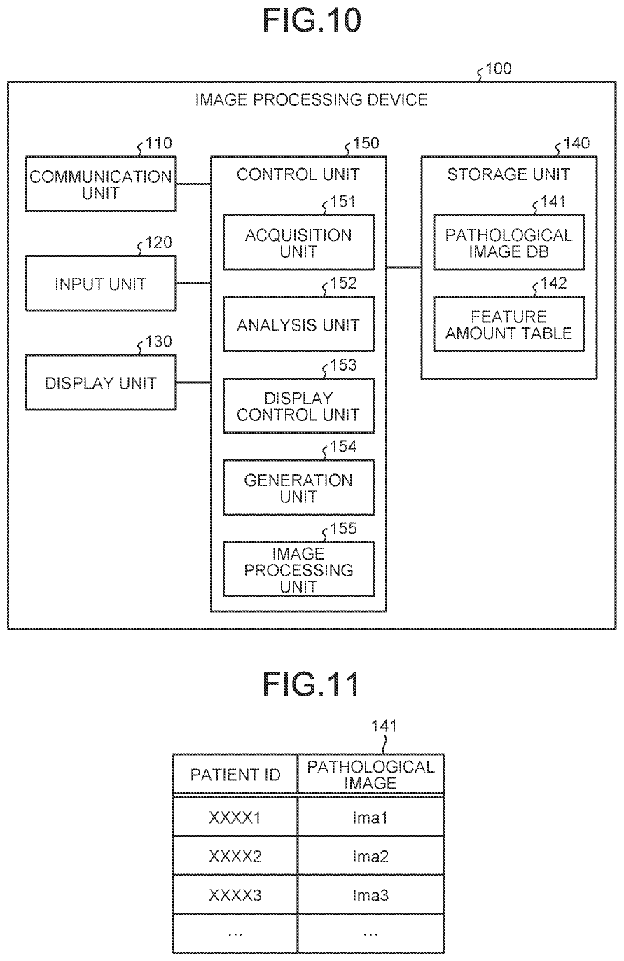

Next, the image processing device 100 according to the present embodiment will be described. FIG. 10 is a diagram illustrating an example of the image processing device according to the present embodiment. As illustrated in FIG. 10, the image processing device 100 includes a communication unit 110, an input unit 120, a display unit 130, a storage unit 140, and a control unit 150.

The communication unit 110 is realized by, for example, a network interface card (NIC) or the like. The communication unit 110 is connected to a network (not illustrated) in a wired or wireless manner, to transmit and receives information to and from the pathology system 10 and the like via the network. The control unit 150 described later transmits and receives information to and from these devices via the communication unit 110.

The input unit 120 is an input device that inputs various types of information to the image processing device 100. An input unit 111 corresponds to a keyboard, a mouse, a touch panel, or the like.

The display unit 130 is a display device that displays information output from the control unit 150. The display unit 130 corresponds to a liquid crystal display, an organic electro luminescence (EL) display, a touch panel, or the like.

The storage unit 140 includes a pathological image data base (DB) 141 and a feature amount table 142. For example, the storage unit 140 is realized by a semiconductor memory device such as a random access memory (RAM) and a flash memory, or a storage device such as a hard disk and an optical disk.

The pathological image DB 141 is a database that stores a plurality of pathological images. FIG. 11 is a diagram illustrating an example of a data structure of the pathological image DB. As illustrated in FIG. 11, the pathological image DB 141 includes a “patient ID” and a “pathological image”. The patient ID is information for uniquely identifying a patient. The pathological image indicates a pathological image stored by the pathologist at the time of diagnosis. The pathological image is transmitted from the server 12. In addition to the patient ID and the pathological image, the pathological image DB 141 may hold information such as the “diagnosis result”, the “grade”, the “tissue type”, the “genetic testing”, the “ultrasonic testing”, and the “medication” described in FIGS. 9A to 9C.

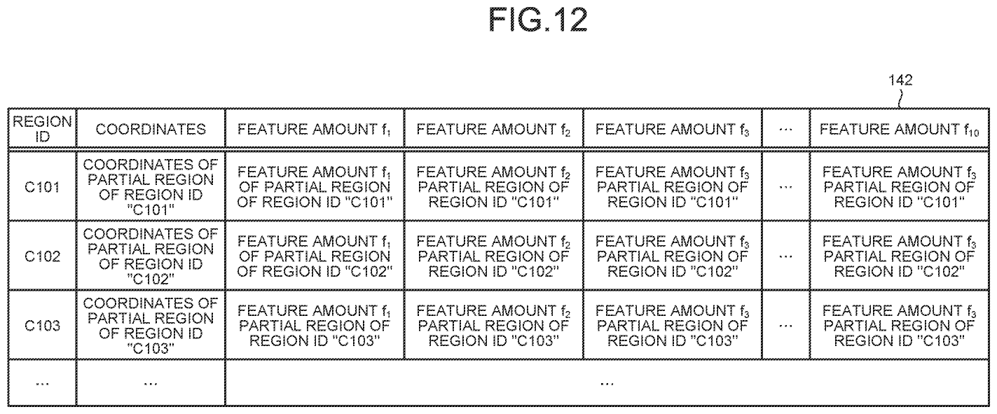

The feature amount table 142 is a table that holds data of feature amounts of partial regions corresponding to cell nuclei and cell membranes extracted from the pathological image. FIG. 12 is a diagram illustrating an example of a data structure of the feature amount table. As illustrated in FIG. 12, the feature amount table 142 associates a region ID, coordinates, and a feature amount. The region ID is information for uniquely identifying a partial region. The coordinates indicate the coordinates (position) of the partial region.

The feature amount is obtained by quantifying characteristics of various patterns including a tissue morphology and a state existing in a pathological image calculated from a partial region. For example, the feature amount corresponds to a feature amount output from a neural network (NN) such as a convolutional neural network (CNN). In addition, the feature amount corresponds to a cell nucleus or a color feature (Luminance, saturation, wavelength, spectrum, and the like), a shape feature (circularity, circumferential length), a density, a distance from a specific morphology, a local feature amount, a structure extraction process (nucleus detection and the like), information obtained by aggregating them (cell density, orientation, and the like), and the like of a cell nucleus. Here, respective feature amounts are indicated by feature amounts f₁ to f₁₀. Note that the feature amount may further include a feature amount fₙ other than the feature amounts f₁ to f₁₀.

The description returns to FIG. 10. The control unit 150 includes an acquisition unit 151, an analysis unit 152, a display control unit 153, a generation unit 154, and an image processing unit 155. The control unit 150 is implemented by, for example, a central processing unit (CPU) or a micro processing unit (MPU) executing a program (an example of an image processing program) stored in the image processing device 100 using a random access memory (RAM) or the like as a work area. Furthermore, the control unit 150 is implemented by, for example, an integrated circuit such as an application specific integrated circuit (ASIC) or a field programmable gate array (FPGA).

The acquisition unit 151 is a processing unit that transmits an acquisition request for the pathological image to the server 12 and acquires the pathological image from the server 12. The acquisition unit 151 registers the acquired pathological image in the pathological image DB 141. The user may operate the input unit 120 to instruct the acquisition unit 151 to acquire the pathological image to be acquired. In this case, the acquisition unit 151 transmits an acquisition request for the instructed pathological image to the server 12 to acquire the instructed pathological image.

The pathological image acquired by the acquisition unit 151 corresponds to a whole slide imaging (WSI). Annotation data indicating part of the pathological image may be attached to the pathological image. The annotation data indicates a tumor region or the like indicated by a pathologist or a researcher. The number of WSIs is not limited to one, and a plurality of WSIs such as serial sections may be included. In addition to the patient ID, information such as the “diagnosis result”, the “grade”, the “tissue type”, the “genetic testing”, the “ultrasonic testing”, and “medication” described in FIGS. 9A to 9C may be attached to the pathological image.

The analysis unit 152 is a processing unit that analyzes the pathological image stored in the pathological image DB 141 and calculates a feature amount. The user may operate the input unit 120 to designate the pathological image to be analyzed.

The analysis unit 152 acquires a pathological image designated by the user from the pathological image DB 141, and extracts a plurality of partial regions (patterns) from the pathological image by performing segmentation on the acquired pathological image. The plurality of partial regions includes individual cells, cell organs (cell nucleus, cell membrane, etc.), and cell morphology by a cell or cell organ aggregation. In addition, the partial region may be a region corresponding to a specific feature possessed in a case where the cell morphology is normal or in a case of a specific disease. Here, the segmentation is a technique of assigning a label of an object of a site in units of pixels from an image. For example, a learned model is generated by causing a convolution neural network to learn an image data set having a correct answer label, and an image (pathological image) desired to be processed is input to the learned model, whereby a label image to which a label of an object class is allocated in units of pixels can be obtained as an output, and a partial region can be extracted for each pixel by referring to the label.

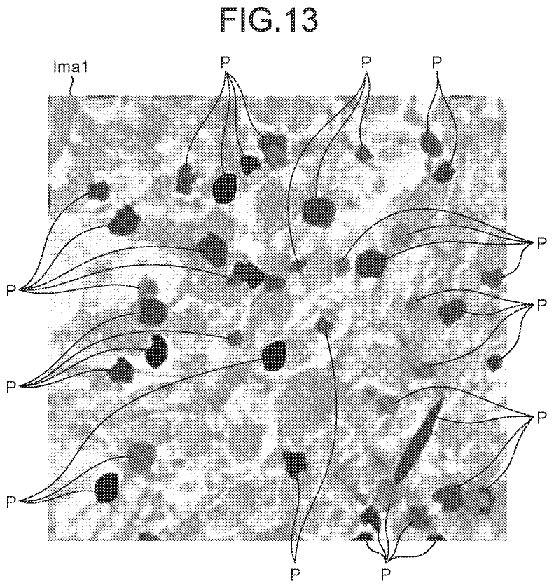

FIG. 13 is a diagram illustrating an example of a partial region extracted from a pathological image. As illustrated in FIG. 13, a plurality of partial regions “P” is extracted from the pathological image Ima1. In the following description, in a case where a plurality of partial regions is not particularly distinguished, they are simply referred to as partial regions. The analysis unit 152 assigns a region ID to a partial region and identifies coordinates of the partial region. The analysis unit 152 registers the coordinates of the partial region in the feature amount table 142 in association with the region ID.

Subsequently, the analysis unit 152 calculates a feature amount from the partial region. For example, the analysis unit 152 calculates a feature amount by inputting the image of the partial region to the CNN. In addition, the analysis unit 152 calculates a color feature (luminance value, dyeing intensity, etc.), a shape feature (circularity, circumferential length, etc.), a density, a distance from a specific morphology, and a local feature amount based on the image of the partial region. Any conventional technique may be used for the process in which the analysis unit 152 calculates the color feature, the shape feature, the density, the distance from the specific morphology, and the local feature amount. The analysis unit 152 registers the feature amounts (for example, the feature amounts f₁ to f₁₀) of the partial region in the feature amount table 142 in association with the region ID.

The analysis unit 152 may perform the above process after receiving an instruction for the pathological image to be analyzed from the user, or may calculate the feature amount of the partial region from a result of analyzing the entire pathological image in advance. Furthermore, the pathology system 10 may analyze the entire pathological image, and the analysis result by the pathology system 10 may be attached to the pathological image, and the analysis unit 152 may calculate the feature amount of the partial region using the analysis result by the pathology system 10.

The display control unit 153 is a processing unit that causes the display unit 130 to display screen information about a pathological image indicating the partial region (various patterns including the tissue morphology) extracted by the analysis unit 152 and receives designation of the partial region. For example, the display control unit 153 acquires the coordinates of each partial region from the feature amount table 142 and reflects the coordinates in the screen information.

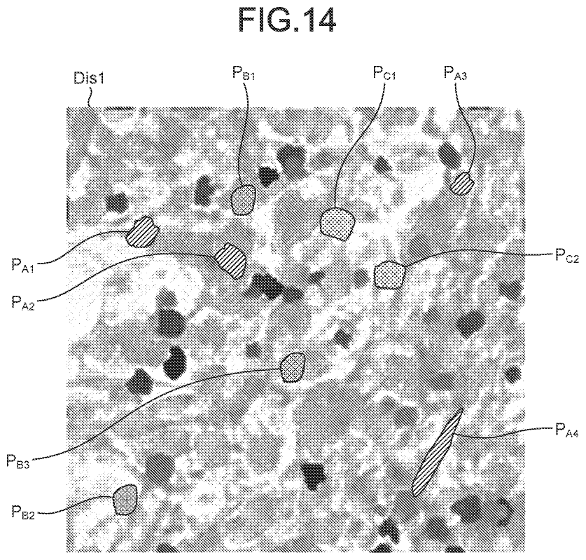

FIG. 14 is a diagram for describing a process by the display control unit. As illustrated in FIG. 14, the display control unit 153 displays the screen information Dis1 on the display unit 130 such that a partial region can be designated. The user operates the input unit 120 to designate some partial regions from the plurality of partial regions and designate a category. For example, it is assumed that partial regions P_{A1}, P_{A2}, P_{A3}, and P_{A4} are selected as the first category. It is assumed that the partial regions P_{B}, P_{B2}, and P_{B3} are selected as the second category. It is assumed that the partial regions P_{C1} and P_{C2} are selected as the third category. In the following description, the partial regions P_{A1}, P_{A2}, P_{A3}, and P_{A4} are appropriately collectively referred to as a partial region “P_{A}”. The partial regions P_{B}, P_{B2}, and P_{B3} are appropriately collectively referred to as a partial region “P_{B}”. The partial regions P_{C1} and P_{C2} are appropriately collectively referred to as a partial region “P_{C}”. The display control unit 153 may display partial regions belonging to the same category in the same color.

In the example illustrated in FIG. 14, the process in which the display control unit 153 receives designation of a partial region is not limited to the above process. For example, in a case where the user designates one partial region, the display control unit 153 may automatically select another partial region similar to the shape of the designated partial region and determine the another partial region as a partial region belonging to the same category.

In the example illustrated in FIG. 14, the case of selecting the partial region extracted by segmentation is described, but the region designated by the user may be a free region or a geometric region drawn by the user. The display control unit 153 may handle an annotation region such as a tumor region designated in advance by a pathologist or a researcher as a designated partial region. In addition, the display control unit 153 may use an extractor for extracting a specific tissue to set a partial region of the tissue extracted by the extractor as a designated partial region.

The display control unit 153 outputs the region ID of the designated partial region and the information about the category of the partial region to the generation unit 154. In the following description, it is assumed that a region ID of a designated partial region is appropriately referred to as a “designated region ID”, and information about a category designated by the user is associated with the designated region ID. Note that the display control unit 153 outputs the first to fourth auxiliary information generated by the generation unit 154 to be described later to the display unit 130 to display it on the display unit 130.

The generation unit 154 is a processing unit that acquires the feature amount corresponding to the designated region ID from the feature amount table 142 and generates auxiliary information about the feature amount of the pathological image. The auxiliary information includes information that enables identification of a feature amount important for expressing a feature of a region desired to be classified or extracted, a distribution of feature amounts, and the like. For example, the generation unit 154 generates the first to fourth auxiliary information as the auxiliary information.

The process in which the generation unit 154 generates the “first auxiliary information” will be described. The generation unit 154 calculates a contribution rate (or importance) when classifying or extracting a partial region of the designated region ID for each category with respect to a plurality of feature amounts (for example, the feature amounts f₁ to f₁₀) calculated from the pathological image, and generates first auxiliary information.

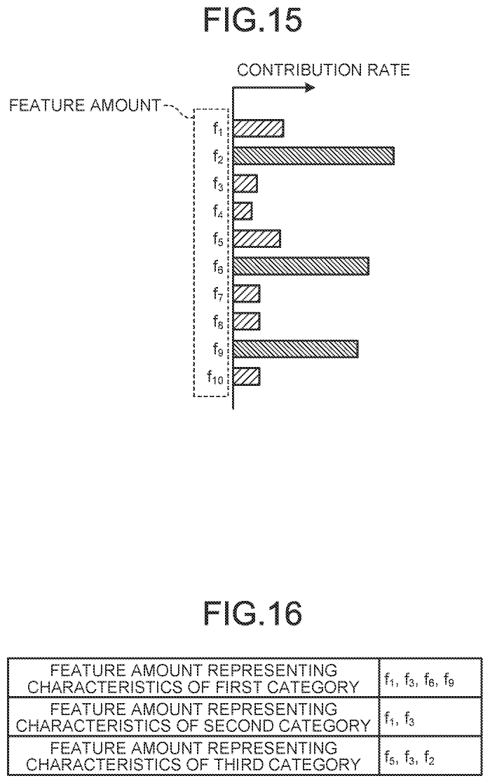

As illustrated in FIG. 14, in a case where the partial region P_{A} of the first category, the partial region P_{B} of the second category, and the partial region P_{C} of the third category are designated, the generation unit 154 calculates contribution rates for classifying the partial regions P_{A}, P_{B}, and P_{C} based on factor analysis, prediction analysis, and the like. For example, as a result of the factor analysis, in a case where the contribution rate of the feature amount f₂ among the feature amounts f₁ to f₁₀ increases, it means that it is appropriate to place a weight on the feature amount f₂ when classifying the partial regions P_{A}, P_{B}, and P_{C}. The generation unit 154 generates the first auxiliary information illustrated in FIG. 15.

FIG. 15 is a diagram illustrating an example of the first auxiliary information. As illustrated in FIG. 15, in the first auxiliary information, the feature amounts f₁ to f₁₀ are associated with the contribution rates. In the example illustrated in FIG. 15, since the contribution rates of the feature amounts f₂, f₆, and f₉ among the feature amounts f₁ to f₁₀ are large, it means that it is appropriate to use the feature amounts f₂, f₆, and f₉ in a case of classifying the partial regions P_{A}, P_{B}, and P_{C}. The generation unit 154 outputs the first auxiliary information to the display control unit 153 to request the display of the first auxiliary information. The display control unit 153 displays the first auxiliary information on the display unit 130. In a case of displaying the first auxiliary information, the display control unit 153 may sort and display the respective feature amounts according to the magnitude of the contribution rate.

The process in which the generation unit 154 generates the “second auxiliary information” will be described. The generation unit 154 compares each feature amount corresponding to the designated region ID with a threshold value set in advance for each feature amount, and performs the process for identifying a feature amount equal to or greater than the threshold value for each category, thereby generating second auxiliary information.

FIG. 16 is a diagram illustrating an example of the second auxiliary information. The generation unit 154 compares the feature amounts f₁ to f₁₀ of the designated region ID corresponding to the first category with the threshold values Th₁ to Th₁₀ of the respective feature amounts. For example, in a case where the feature amount f₁ is equal to or more than the threshold value Th₁, the feature amount f₃ is equal to or more than the threshold value Th₃, the feature amount f₆ is equal to or more than the threshold value Th₆, and the feature amount f₉ is equal to or more than the threshold value Th₉, the generation unit 154 sets the feature amounts f₁, f₃, f₆, and f₉ as the feature amounts representing the characteristics of the first category.

The generation unit 154 compares the feature amounts f₁ to f₁₀ of the designated region ID corresponding to the second category with the threshold values Th₁ to Th₁₀ of the respective feature amounts. For example, in a case where the feature amount f₁ is equal to or more than the threshold value Th₁ and the feature amount f₃ is equal to or more than the threshold value Th₃, the generation unit 154 sets the feature amounts f₁ and f₃ as the feature amounts representing the characteristics of the second category.

The generation unit 154 compares the feature amounts f₁ to f₁₀ of the designated region ID corresponding to the third category with the threshold values Th₁ to Th₁₀ of the respective feature amounts. For example, in a case where the feature amount f₅ is equal to or more than the threshold value Th₅, the feature amount f₃ is equal to or more than the threshold value Th₃, and the feature amount f₂ is equal to or more than the threshold value Th₂, the generation unit 154 sets the feature amounts f₅, f₃, and f₂ as the feature amounts representing the characteristics of the third category.

The generation unit 154 performs the above process to generate the second auxiliary information illustrated in FIG. 16. In the example illustrated in FIG. 16, in a case where a partial region of the first category is extracted, it means that the feature amounts f₁, f₃, f₆, and f₉ are suitable. In a case where a partial region of the second category is extracted, it means that the feature amounts f₁ and f₃ are suitable. In a case where a partial region of the third category is extracted, this means that the feature amounts f₅, f₃, and f₂ are suitable.

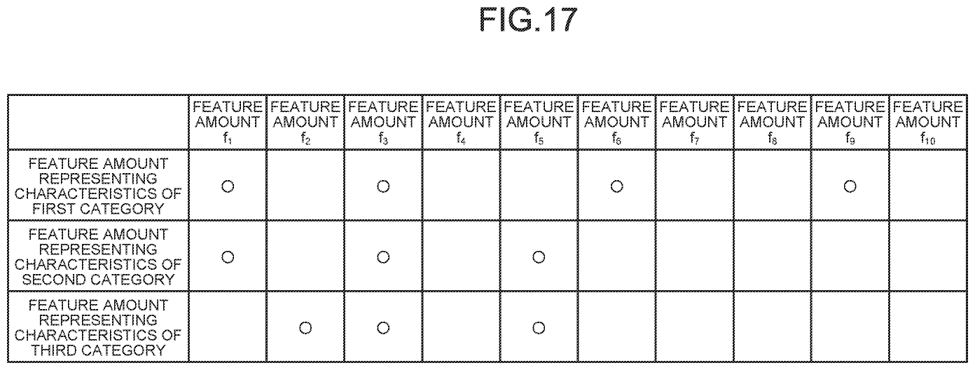

The generation unit 154 outputs the second auxiliary information to the display control unit 153 to request the display of the second auxiliary information. The display control unit 153 displays the second auxiliary information on the display unit 130. Note that the display control unit 153 may display the second auxiliary information on the display unit 130 in the form of a table illustrated in FIG. 17.

FIG. 17 is a diagram illustrating another display example of the second auxiliary information. In the row of the feature amounts indicating the characteristics of the first category of the display example illustrated in FIG. 17, the feature amounts f₁, f₃, f₆, and f₉ are indicated by circles, indicating that the feature amounts f₁, f₃, f₆, and f₉ are suitable. In the row of the feature amounts indicating the characteristics of the second category, the feature amounts f₁, f₃, and f₅ are indicated by circles, indicating that the feature amounts f₁, f₃, and f₅ are suitable. In the row of the feature amounts indicating the characteristics of the third category, the feature amounts f₃, and f₂ are indicated by circles, indicating that the feature amounts f₅, f₃, and f₂ are suitable. As compared with the display of FIG. 16, a suitable feature amount and an unsuitable feature amount can be easily grasped in FIG. 17.

The process in which the generation unit 154 generates the “third auxiliary information” will be described. The generation unit 154 generates third auxiliary information in which the distribution of respective partial regions is disposed in a feature space having the first feature amount fᵢ and the second feature amount fⱼ as axes among the feature amounts f₁ to f₁₀. The first feature amount fᵢ and the second feature amount fⱼ may be set in advance, or the feature amounts corresponding to the higher contribution rate may be set as the first feature amount fᵢ and the second feature amount fⱼ based on the contribution rate calculated in the case of generating the first auxiliary information in FIG. 15.

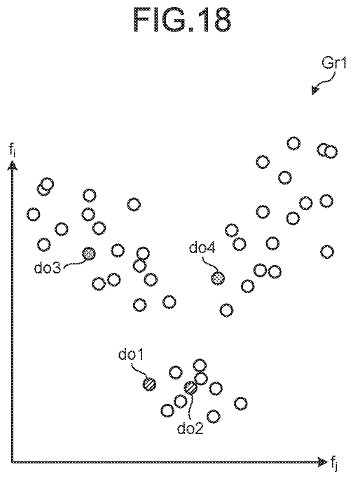

FIG. 18 is a diagram illustrating an example of the third auxiliary information. A vertical axis of a feature space Gr1 illustrated in FIG. 18 is an axis corresponding to the first feature amount fᵢ, and a horizontal axis is an axis corresponding to the second feature amount fⱼ. The generation unit 154 refers to the feature amount table 142, identifies the first feature amount fᵢ and the second feature amount fⱼ of each partial region, and plots a point corresponding to each partial region on the feature space Gr1. In addition, the generation unit 154 sets the point of the partial region corresponding to the designated region ID among the points corresponding to the respective partial regions to be identifiable.

For example, in a case where the partial region P_{A1} of the first category designated in FIG. 14 corresponds to the point do1 in FIG. 18, the generation unit 154 disposes the point do1 in the first color indicating that the partial region P_{A1} belongs to the first category. In a case where the partial region P_{A2} of the first category designated in FIG. 14 corresponds to the point dot in FIG. 18, the point do1 is disposed in the first color indicating that the partial region P_{A2} belongs to the first category.

In a case where the partial region PB1 of the second category designated in FIG. 14 corresponds to the point do3 in FIG. 18, the generation unit 154 disposes the point do3 in the second color indicating that the partial region PB1 belongs to the second category. In a case where the partial region P_{C1} of the third category designated in FIG. 14 corresponds to the point do4 in FIG. 18, the point do4 is disposed in the third color indicating that the partial region P_{C1} belongs to the third category. It is assumed that the first color, the second color, and the third color are different colors.

The generation unit 154 outputs the third auxiliary information to the display control unit 153 to request the display of the third auxiliary information. The display control unit 153 displays the third auxiliary information on the display unit 130. Note that, in the multidimensional feature amount, the generation unit 154 may calculate the low-dimensional feature amount obtained using dimension reduction such as principal component analysis or TSNE, and plot points corresponding to respective partial regions in the feature space of the low-dimensional feature amount.

The process in which the generation unit 154 generates the “fourth auxiliary information” will be described. Based on the feature amount table 142, the generation unit 154 generates a histogram of each of the feature amounts f₁ to f₁₀ as the fourth auxiliary information.

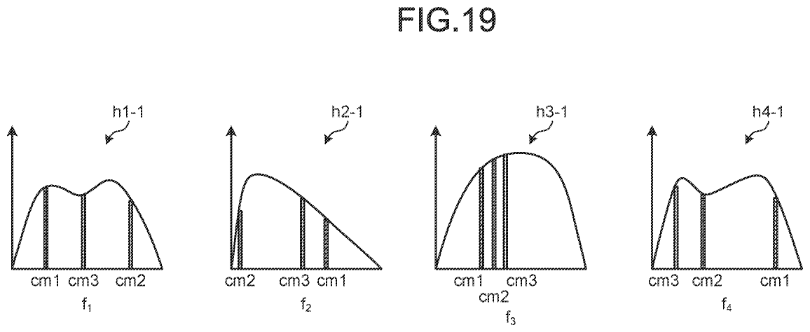

FIG. 19 is a diagram illustrating an example of the fourth auxiliary information. FIG. 19 illustrates histograms h1-1 to h4-1 of the respective feature amounts as an example. The generation unit 154 may set the frequency of the class value corresponding to the feature amount of the partial region corresponding to the designated region ID to be identifiable in each histogram.

The histogram h1-1 is a histogram corresponding to the feature amount f₁. In a case where the feature amount f₁ of the partial region P_{A1} of the first category corresponds to the class value cm1, the generation unit 154 sets the color of the frequency corresponding to the class value cm1 to the first color. In a case where the feature amount f₁ of the partial region P_{B1} of the second category corresponds to the class value cm2, the generation unit 154 sets the color of the frequency corresponding to the class value cm2 to the second color. In a case where the feature amount f₁ of the partial region P_{C1} of the third category corresponds to the class value cm3, the generation unit 154 sets the color of the frequency corresponding to the class value cm3 to the third color.

The histogram h2-1 is a histogram corresponding to the feature amount f₂. In a case where the feature amount f₂ of the partial region P_{A1} of the first category corresponds to the class value cm1, the generation unit 154 sets the color of the frequency corresponding to the class value cm1 to the first color. In a case where the feature amount f₂ of the partial region P_{B1} of the second category corresponds to the class value cm2, the generation unit 154 sets the color of the frequency corresponding to the class value cm2 to the second color. In a case where the feature amount f₂ of the partial region P_{C1} of the third category corresponds to the class value cm3, the generation unit 154 sets the color of the frequency corresponding to the class value cm3 to the third color.

The histogram h3-1 is a histogram corresponding to the feature amount f₃. In a case where the feature amount f₃ of the partial region P_{A1} of the first category corresponds to the class value cm1, the generation unit 154 sets the color of the frequency corresponding to the class value cm1 to the first color. In a case where the feature amount f₃ of the partial region P_{B1} of the second category corresponds to the class value cm2, the generation unit 154 sets the color of the frequency corresponding to the class value cm2 to the second color. In a case where the feature amount f₃ of the partial region P_{C1} of the third category corresponds to the class value cm3, the generation unit 154 sets the color of the frequency corresponding to the class value cm3 to the third color.

The histogram h4-1 is a histogram corresponding to the feature amount f₄. In a case where the feature amount f₄ of the partial region P_{A1} of the first category corresponds to the class value cm1, the generation unit 154 sets the color of the frequency corresponding to the class value cm1 to the first color. In a case where the feature amount f₄ of the partial region P_{B1} of the second category corresponds to the class value cm2, the generation unit 154 sets the color of the frequency corresponding to the class value cm2 to the second color. In a case where the feature amount f₄ of the partial region P_{C1} of the third category corresponds to the class value cm3, the generation unit 154 sets the color of the frequency corresponding to the class value cm3 to the third color.

Although not illustrated, the generation unit 154 similarly generates histograms corresponding to the feature amounts f₅ to f₁₀. The generation unit 154 outputs the fourth auxiliary information to the display control unit 153 to request the display of the fourth auxiliary information. The display control unit 153 displays the fourth auxiliary information on the display unit 130.

Here, the display control device 153 may display all the 1st to 4th auxiliary information on the display unit 130, or may display only part of the auxiliary information. Furthermore, the user may operate the input unit 120 to designate the auxiliary information to be displayed. In the following description, in a case where the first to fourth auxiliary information is not particularly distinguished, they are simply referred to as auxiliary information.

In addition, after referring to the auxiliary information, the user may operate an input unit 130, refer to screen information Dis1 illustrated in FIG. 14 again, and select the partial region and the category of the partial region again. In a case where the partial region and the category of the partial region are reselected, the display control unit 153 outputs a new designated region ID to the generation unit 154. The generation unit 154 generates new auxiliary information based on the new designated region ID, and the display control unit 153 outputs the new auxiliary information to the display unit 130 to display it on the display unit 130. The display control unit 153 and the generation unit 154 repeatedly perform the above process each time the user reselects the partial region and the category of the partial region.

The description returns to FIG. 10. The image processing unit 155 is a processing unit that performs various types of image processes on a pathological image in a case where designation of the pathological image is received from the user. For example, based on the parameter, the image processing unit 155 performs the process of classifying a partial region included in the pathological image according to the feature amount, the process of extracting a partial region having a specific feature amount, and the like. The parameter is set by the user who refers to the auxiliary information.

In a case where the image processing unit 155 performs the image process of classifying the partial region included in the pathological image according to the feature amount, the user operates the input unit 120 to set, as parameters, the feature amount (some feature amounts of the feature amounts f₁ to f₁₀) to be used at the time of classification, the importance of the feature amount, and the like.

In a case where the image processing unit 155 performs the image process of extracting a partial region having a specific feature amount from partial regions included in the pathological image, the user operates the input unit 120 to set, as parameters, the feature amount (some feature amounts of the feature amounts f₁ to f₁₀) to be used at the time of extraction, the threshold value for each feature amount at the time of extraction, and the like.

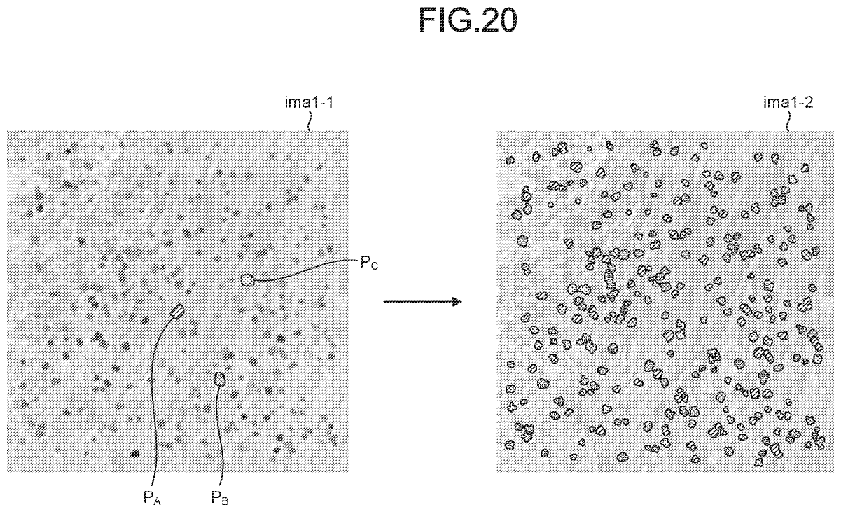

Here, an example of a processing result by the image processing unit 155 will be described. FIGS. 20, 21, and 22 are diagrams for explaining an example of a classification process performed by the image processing unit 155. FIG. 20 will be described. A pathological image Ima1-1 is a pathological image before the classification process is performed. In the pathological image Ima1-1, the user designates the partial region P_{A} as the first category, designates the partial region P_{B} as the second category, and selects the partial region P_{C} as the third category. The partial region P_{A} is indicated by the first color. The partial region P_{B} is indicated by the second color. The partial region P_{C} is indicated in the third color.

The image processing unit 155 classifies each partial region included in the pathological image Ima1-1 into one of the first category, the second category, and the third category based on the parameter set to the user. The classification result is illustrated in a pathological image Ima1-2. In the pathological image Ima1-2, each partial region indicated by the first color is a partial region classified into the first category. Each partial region indicated by the second color is a partial region classified into the second category. Each partial region indicated by the third color is a partial region classified into the third category. The image processing unit 155 may output the pathological image Ima1-2 as the classification result to the display unit 130 to display it on the display unit 130.

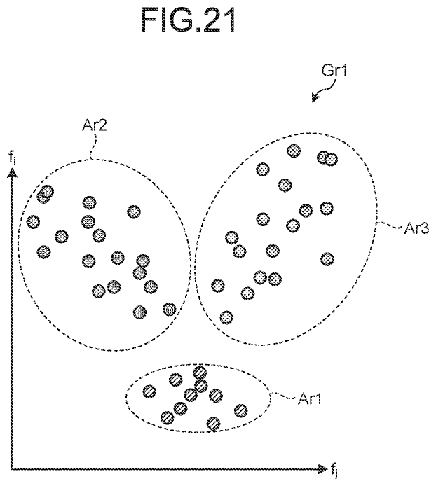

FIG. 21 will be described. FIG. 21 illustrates a case where the image processing unit 155 plots partial regions classified into the first category, the second category, and the third category in the feature space Gr1 according to the feature amount. The vertical axis of the feature space Gr1 is an axis corresponding to the first feature amount fᵢ, and the horizontal axis is an axis corresponding to the second feature amount fⱼ. In the example illustrated in FIG. 21, the partial regions classified into the first category are located in a region Ar1. The partial region classified into the second category is located in a region Art. The partial region classified into the third category is located in a region Ar3. The image processing unit 155 may output the information about the feature space Gr1 illustrated in FIG. 21 to the display unit 130 to display it on the display unit 130.

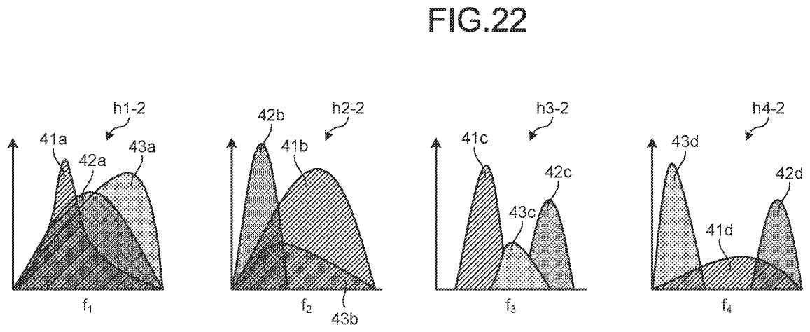

FIG. 22 will be described. In FIG. 22, histograms h1-2 to h4-2 of the respective feature amounts are illustrated. The image processing unit 155 generates histograms h1-2 to h4-2 by making the distribution of the feature amounts of the partial regions classified into the first category, the distribution of the feature amounts of the partial regions classified into the second category, and the distribution of the feature amounts of the partial regions classified into the third category identifiable with each other.

The histogram h1-2 is a histogram corresponding to the feature amount f₁. In the histogram h1-2, a distribution 41a is a distribution of the feature amounts of the partial regions classified into the first category. A distribution 42a is a distribution of the feature amounts of the partial regions classified into the second category. A distribution 43a is a distribution of the feature amounts of the partial regions classified into the third category.

The histogram h2-2 is a histogram corresponding to the feature amount f₂. In the histogram h2-2, the distribution 41b is a distribution of the feature amounts of the partial regions classified into the first category. The distribution 42b is a distribution of the feature amounts of the partial regions classified into the second category. The distribution 43b is a distribution of the feature amounts of the partial regions classified into the third category.

The histogram h3-2 is a histogram corresponding to the feature amount f₃. In the histogram h3-2, the distribution 41c is a distribution of the feature amounts of the partial regions classified into the first category. The distribution 42c is a distribution of the feature amounts of the partial regions classified into the second category. The distribution 43c is a distribution of the feature amounts of the partial regions classified into the third category.

The histogram h4-2 is a histogram corresponding to the feature amount f₄. In the histogram h4-2, the distribution 41d is a distribution of the feature amounts of the partial regions classified into the first category. The distribution 42d is a distribution of the feature amounts of the partial regions classified into the second category. The distribution 43d is a distribution of the feature amounts of the partial regions classified into the third category.

Although not illustrated, the image processing unit 155 similarly generates histograms corresponding to the feature amounts f₅ to f₁₀. The image processing unit 155 may output the information about the histograms h1-2 to h4-2 illustrated in FIG. 22 to the display unit 130 to display it on the display unit 130.

### 4. Processing Procedure

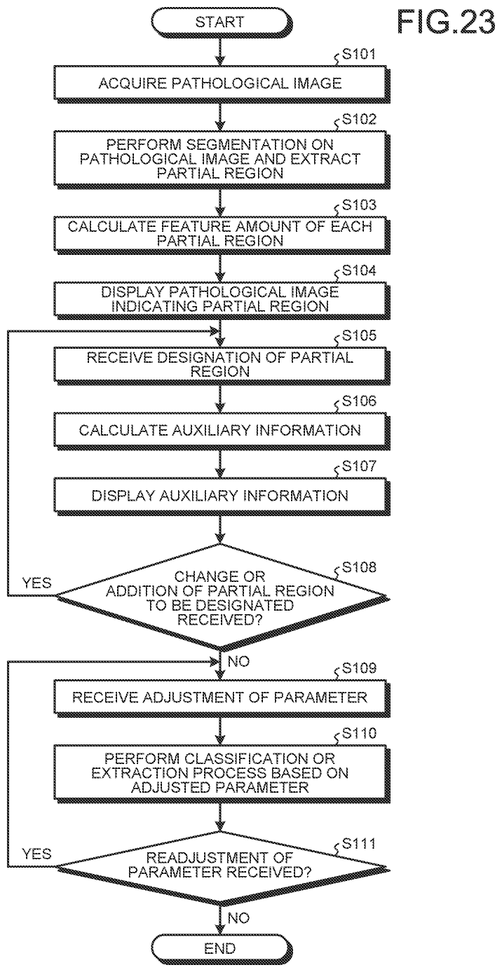

FIG. 23 is a flowchart illustrating a processing procedure of the image processing device 100 according to the present embodiment. As illustrated in FIG. 23, the acquisition unit 151 of the image processing device 100 acquires a pathological image (step S101). The analysis unit 152 of the image processing device 100 performs segmentation on the pathological image and extracts a partial region (step S102).

The analysis unit 152 calculates a feature amount of each partial region (step S103). The display control unit 153 displays the pathological image indicating the partial region on the display unit 130 (step S104). The display control unit 153 receives designation of the partial region (step S105).

The generation unit 154 of the image processing device 100 generates auxiliary information (step S106). The display control unit 153 displays the auxiliary information on the display unit 130 (step S107).

In a case of receiving a change or an addition of the partial region to be designated (step S108, Yes), the image processing device 100 advances the process to step S105. On the other hand, in a case of not receiving the change or addition of the partial region to be designated (step S108, No), the image processing device 100 advances the process to step S109.

The image processing unit 155 of the image processing device 100 receives adjustment of parameters (step S109). The image processing unit 155 performs the classification or extraction process based on the adjusted parameters (step S110).

In a case where the re-adjustment of the parameter is received (step S111, Yes), the image processing device 100 advances the process to step S109. In a case where re-adjustment of the parameters is not received (step S111, No), the image processing device 100 ends the process.

### 5. Another Process

The image processing device 100 may generate, as the auxiliary information, information capable of grasping the situation of a plurality of partial regions in the pathological image, such as the situation of the entire pathological image, and display the auxiliary information.

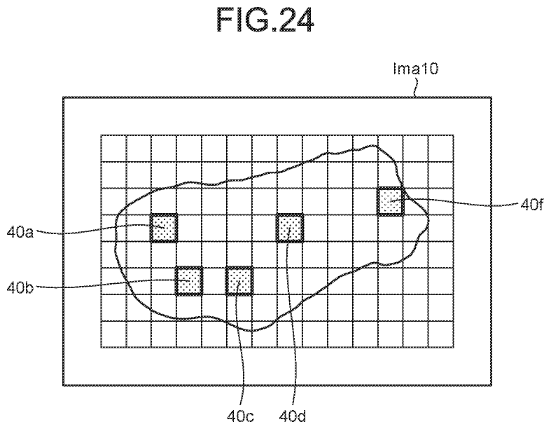

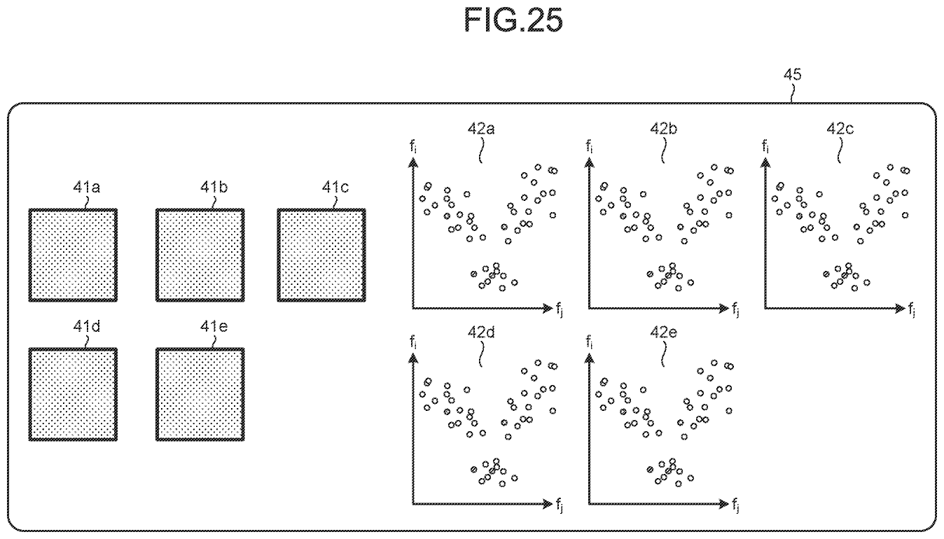

FIGS. 24 and 25 are diagrams for explaining another process by the image processing device 100. FIG. 24 will be described. The display control unit 153 of the image processing device 100 displays a pathological image Ima10 divided into a plurality of regions of interest (ROIs). The user operates the input unit 120 to designate a plurality of ROIs. In the example illustrated in FIG. 24, a case where ROIs 40a, 40b, 40c, 40d, and 40e are designated is illustrated. When receiving the designation of the ROI, the display control unit 153 displays the screen information illustrated in FIG. 25.

FIG. 25 will be described. The display control unit 153 displays the enlarged ROI images 41a to 41e in screen information 45. The image 41a is an enlarged image of the ROI 40a. The image 41b is an enlarged image of the ROI 40b. The image 41c is an enlarged image of the ROI 40c. The image 41d is an enlarged image of the ROI 40d. The image 41e is an enlarged image of the ROI 40e.

The analysis unit 152 of the image processing device 100 extracts partial regions from the ROI 40a and calculates a feature amount of each partial region in the same manner as the above processing. The generation unit 154 of the image processing device 100 generates auxiliary information 42a based on the feature amounts of respective partial regions of the ROI 40a and sets the auxiliary information in the screen information 45. For example, the auxiliary information 42a may be the third auxiliary information described with reference to FIG. 18, or may be another auxiliary information. The generation unit 154 also generates auxiliary information 42b to 42e based on the feature amounts of respective partial regions of the ROIs 40b to 40e, respectively, and sets the auxiliary information in the screen information 45.

The user can grasp the features of the entire pathological image by referring to the screen information 45, and can use the features for parameter adjustment in a case where the image process is performed.

### 6. Effects of Image Processing Device According to Present Embodiment

The image processing device 100 according to the present embodiment extracts a plurality of partial regions from a pathological image, and generates auxiliary information indicating a feature amount effective in a case of classifying or extracting a partial image with respect to a plurality of feature amounts calculated from the pathological image when designation of the partial region is received. In a case of receiving the setting of the parameter from the user who has referred to the auxiliary information, the image processing device 100 performs the image process on the pathological image using the received parameter. As a result, the feature amount obtained by quantifying the appearance feature of the morphology can be appropriately displayed by the auxiliary information, and adjustment of the parameter of the image process can be facilitated. For example, the “macroscopic and visible feature” of a specialist such as a pathologist can be easily associated with the “calculated quantitative feature”.

The image processing device 100 calculates a contribution rate when classifying the plurality of designated partial regions, and generates and displays, as auxiliary information, information in which the feature amount and the contribution rate are associated with each other. By referring to such auxiliary information, the user can easily grasp which feature amount should be emphasized to set the parameter in a case of classifying the plurality of partial regions for each category.

The image processing device 100 selects some feature amounts based on the magnitudes of the plurality of feature amounts calculated from the plurality of designated partial regions, and generates the selected feature amount as auxiliary information. By referring to such auxiliary information, the user can easily grasp the feature amount to be used in a case of extracting a partial region having the category same as that of the designated partial region.

The image processing device 100 performs segmentation on the pathological image and extracts a plurality of partial regions. As a result, the user can easily designate the region corresponding to the cell morphology included in the pathological image.

The image processing device 100 displays all the partial regions included in the pathological image, and receives selection of a plurality of partial regions among all the partial regions. As a result, the user can easily select the partial region to be used in the creation of the auxiliary information.

The image processing device 100 performs the factor analysis, prediction analysis, or the like to calculate a contribution rate. As a result, it is possible to calculate the feature amount effective in a case where the partial region is appropriately classified for each of the designated different categories.

The image processing device 100 generates a feature space corresponding to some feature amounts, and identifies a position in the feature amount space corresponding to the partial region designation of which was received based on the feature amount of the partial region designation of which was received. As a result, the user can easily grasp the position in the feature space with respect to the designated partial region.

The image processing device 100 identifies a feature amount having a high contribution rate and generates a feature space of the identified feature amount. As a result, the user can grasp the distribution of the designated partial regions in the feature space of the feature amount with a high contribution rate.

In a case where a plurality of ROIs is designated for the entire pathological image, the image processing device 100 generates auxiliary information based on the feature amount of the partial region included in each ROI. As a result, it is possible to grasp the features of the entire pathological image and to use the features for parameter adjustment when the image process is performed.

### 3. Hardware Configuration

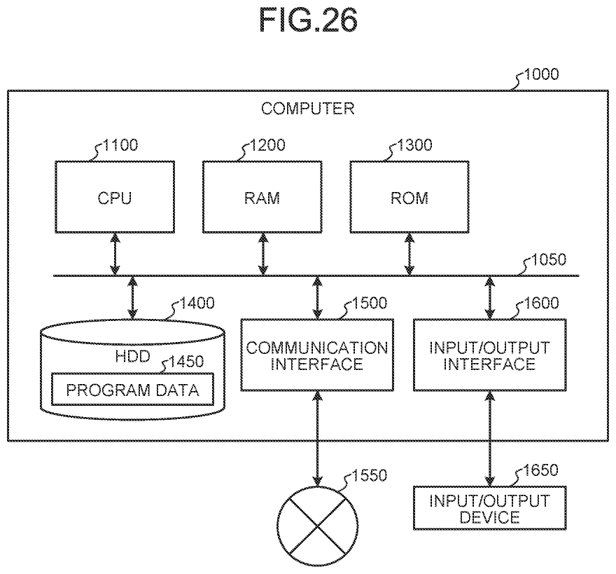

The image processing device according to each embodiment described above is implemented by, for example, a computer 1000 having a configuration as illustrated in FIG. 26. Hereinafter, an imaging system 100 according to the embodiments will be described as an example. FIG. 26 is a hardware configuration diagram illustrating an example of the computer 1000 that implements the functions of the image processing device. The computer 1000 includes a CPU 1100, a RAM 1200, a read only memory (ROM) 1300, a hard disk drive (HDD) 1400, a communication interface 1500, and an input/output interface 1600. Respective units of the computer 1000 are connected by a bus 1050.

The CPU 1100 operates based on a program stored in the ROM 1300 or the HDD 1400, and controls each unit. For example, the CPU 1100 develops a program stored in the ROM 1300 or the HDD 1400 in the RAM 1200, and executes processing corresponding to various programs.

The ROM 1300 stores a boot program such as a basic input output system (BIOS) executed by the CPU 1100 when the computer 1000 is activated, a program depending on hardware of the computer 1000, and the like.

The HDD 1400 is a computer-readable recording medium that non-transiently records programs executed by the CPU 1100, data used by the programs, and the like. Specifically, the HDD 1400 is a recording medium that records an information processing program according to the present disclosure which is an example of program data 1450.

The communication interface 1500 is an interface for the computer 1000 to be connected to an external network 1550 (for example, the Internet). For example, the CPU 1100 receives data from another device or transmits data generated by the CPU 1100 to another device via the communication interface 1500.

The input/output interface 1600 is an interface that connects an input/output device 1650 and the computer 1000. For example, the CPU 1100 receives data from an input device such as a keyboard and a mouse via the input/output interface 1600. In addition, the CPU 1100 transmits data to an output device such as a display, a speaker, or a printer via the input/output interface 1600. Furthermore, the input/output interface 1600 may function as a media interface that reads a program or the like recorded in a predetermined recording medium (medium). The medium is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

Furthermore, the computer 1000 is connected to a millimeter wave radar or a camera module (corresponding to an image generation unit 107 or the like) via the input/output interface 1600.

For example, in a case where the computer 1000 functions as the image processing device 100 according to the embodiments, the CPU 1100 of the computer 1000 executes the image processing program loaded on the RAM 1200 to implement the functions of the acquisition unit 151, the analysis unit 152, the display control unit 153, the generation unit 154, the image processing unit 155, and the like. Further, the HDD 1400 stores an image processing program or the like according to the present disclosure. The CPU 1100 reads the program data 1450 from the HDD 1400 and executes the program data, but as another example, the program may be acquired from another device via the external network 1550.

### 4. Conclusion

The image processing device includes a generation unit and an image processing unit. In a case of receiving designation of a plurality of partial regions extracted from a pathological image and corresponding to a cell morphology, the generation unit generates auxiliary information indicating information about a feature amount effective when classifying or extracting the plurality of partial regions with respect to the plurality of feature amounts calculated from the image. In a case of receiving setting information about an adjustment item according to the auxiliary information, the image processing unit performs the image process on the image using the setting information. As a result, the feature amount obtained by quantifying the appearance feature of the morphology can be appropriately displayed by the auxiliary information, and adjustment of the parameter of the image process can be facilitated. For example, it is possible to easily associated the “feature based on knowledge of a specialist such as a pathologist” with the “calculated quantitative feature”.

The generation unit calculates a contribution rate when classifying the plurality of designated partial regions, and generates, as the auxiliary information, information in which the feature amount and the contribution rate are associated with each other. By referring to such auxiliary information, the user can easily grasp which feature amount should be emphasized to set the parameter in a case of classifying the plurality of partial regions for each category.

The generation unit selects some feature amounts based on the magnitudes of the plurality of feature amounts calculated from the plurality of designated partial regions, and generates information about the selected feature amounts as the auxiliary information. By referring to such auxiliary information, the user can easily grasp the feature amount to be used in a case of extracting a partial region having the category same as that of the designated partial region.

The image processing device performs segmentation on the image and extracts the plurality of partial regions. As a result, the user can easily designate the region corresponding to the cell morphology included in the pathological image.

The image processing device further includes a display control unit that displays all partial regions extracted by the analysis unit and receives designation of a plurality of partial regions among all the partial regions. The display control unit further displays the auxiliary information. As a result, the user can easily select the partial region to be used in the creation of the auxiliary information.

The generation unit performs the factor analysis or the prediction analysis to calculate the contribution rate. As a result, it is possible to calculate the feature amount effective in a case where the partial region is appropriately classified for each of the designated different categories.

The generation unit generates a feature space corresponding to some feature amounts, and identifies a position in the feature amount space corresponding to the partial region designation of which was received based on a feature amount of a partial region designation of which was received. As a result, the user can easily grasp the position in the feature space with respect to the designated partial region.

The generation unit identifies a feature amount having a high contribution rate and generates a feature space of the identified feature amount. As a result, the user can grasp the distribution of the designated partial regions in the feature space of the feature amount with a high contribution rate.

In a case where a plurality of regions is designated for the pathological image, the generation unit generates auxiliary information for each of the plurality of regions. As a result, it is possible to grasp the features of the entire pathological image and to use the features for parameter adjustment when the image process is performed.

### REFERENCE SIGNS LIST

1 DIAGNOSIS SUPPORT SYSTEM
10 PATHOLOGY SYSTEM
11 MICROSCOPE
12 SERVER
13 DISPLAY CONTROL DEVICE
14 DISPLAY DEVICE
100 IMAGE PROCESSING DEVICE
110 COMMUNICATION UNIT
120 INPUT UNIT
130 DISPLAY UNIT
140 STORAGE UNIT
141 PATHOLOGICAL IMAGE DB
142 FEATURE AMOUNT TABLE
150 CONTROL UNIT
151 ACQUISITION UNIT
152 ANALYSIS UNIT
153 DISPLAY CONTROL UNIT
154 GENERATION UNIT
155 IMAGE PROCESSING UNIT

1. An image processing device including:
in a case where designation of a plurality of partial regions corresponding to a cell morphology is received, the plurality of partial regions being extracted from a pathological image, a generation unit that generates auxiliary information indicating information about a feature amount effective when a plurality of partial regions is classified or extracted with respect to a plurality of feature amounts calculated from the image; and
in a case where setting information about an adjustment item according to the auxiliary information is received, an image processing unit that performs an image process on the image using the setting information.

2. The image processing device according to claim 1, wherein the generation unit calculates a contribution rate when the plurality of designated partial regions is classified, and generates, as the auxiliary information, information in which the feature amount and the contribution rate are associated with each other.

3. The image processing device according to claim 1, wherein the generation unit selects some feature amounts based on magnitudes of a plurality of feature amounts calculated from a plurality of designated partial regions, and generates information about the selected feature amounts as the auxiliary information.

4. The image processing device according to claim 1, further including: an analysis unit that performs segmentation on the image and extracts the plurality of partial regions.

5. The image processing device according to claim 4, further including: a display control unit that displays all partial regions extracted by the analysis unit and receives designation of a plurality of partial regions among all the partial regions.

6. The image processing device according to claim 5, wherein the display control unit further displays the auxiliary information.

7. The image processing device according to claim 2, wherein the generation unit performs a factor analysis or a prediction analysis to calculate the contribution rate.

8. The image processing device according to claim 5, wherein the generation unit generates a feature space corresponding to some feature amounts, and identifies a position in the feature amount space corresponding to a partial region designation of which was received based on a feature amount of the partial region designation of which was received.

9. The image processing device according to claim 6, wherein the generation unit identifies a feature amount having a high contribution rate and generates a feature space of the identified feature amount.

10. The image processing device according to claim 1, wherein in a case where a plurality of regions is designated for the pathological image, the generation unit generates auxiliary information for each of the plurality of regions.

11. An image processing method executed by a computer, the method including:
in a case where designation of a plurality of partial regions corresponding to a cell morphology is received, the plurality of partial regions being extracted from a pathological image, generating auxiliary information indicating information about a feature amount effective when a plurality of partial regions is classified or extracted with respect to a plurality of feature amounts calculated from the image; and in a case where setting information about an adjustment item according to the auxiliary information is received, performing an image process on the image using the setting information.

12. An image processing program for causing a computer to function as:
in a case where designation of a plurality of partial regions corresponding to a cell morphology is received, the plurality of partial regions being extracted from a pathological image, a generation unit that generates auxiliary information indicating information about a feature amount effective when a plurality of partial regions is classified or extracted with respect to a plurality of feature amounts calculated from the image; and
in a case where setting information about an adjustment item according to the auxiliary information is received, an image processing unit that performs an image process on the image using the setting information.

13. A diagnosis support system including: a medical image acquisition device and software used for processing a medical image corresponding to an object imaged by the medical image acquisition device, wherein
the software causes an image processing device to
in a case where designation of a plurality of partial regions corresponding to a cell morphology is received, the plurality of partial regions being extracted from a pathological image, generate auxiliary information indicating information about a feature amount effective when a plurality of partial regions is classified or extracted with respect to a plurality of feature amounts calculated from the image; and
in a case where setting information about an adjustment item according to the auxiliary information is received, perform an image process on the image using the setting information.
